(19) United States
(12) Patent Application Publication
(10) Pub. No.: US 2026/0151215 A1
(43) Pub. Date: Jun. 4, 2026

(54) INTRAORAL TOMOGRAPHIC SCANNING APPARATUS

(71) Applicant: Dental Imaging Technologies Corporation, Quakertown, PA (US)

(72) Inventors: Victor C. Wong, Pittsford, NY (US); Xiaodong Tao, Pittsford, NY (US); Levon O. Vogelsang, Webster, NY (US); Wei Ye, Shanghai (CN)

(73) Assignee: Dental Imaging Technologies Corporation, Quakertown, PA (US)

(21) Appl. No.: 18/966,340

(22) Filed: Dec. 3, 2024

(51) Int. Cl. A61C 9/00; A61B 5/00

(52) U.S. Cl. CPC A61C 9/0086; A61C 9/0053; A61B 5/0066; A61B 5/0095

(57) ABSTRACT

A method for scanning an intraoral feature of a patient repeats a sequence of energizing a hand-held scanner to direct a penetrating signal for characterizing a surface contour and condition of at least a portion of underlying tissue of the intraoral feature. Returned signal energy from each of the plurality of adjacent scanned locations is processed to obtain tomographic image content and to reconstruct, from the obtained tomographic image content, a 3-D model of the intraoral feature. Further processing identifies one or more normal or abnormal conditions for intraoral tissue at each of the plurality of adjacent scanned locations. Simultaneously during the scanning, the method repeats a sequence of: displaying the reconstructed 3-D model and updating the reconstructed 3-D model from the obtained tomographic image content and reporting the one or more normal or abnormal conditions identified at scanned locations of the intraoral feature.

### FIELD OF THE INVENTION

The present application is directed to intraoral scanning and more particularly to apparatus for generating and displaying depth-resolved image content related to the condition of intraoral features on and below the tissue surface.

### BACKGROUND OF THE INVENTION

Hand-held scanners have brought about significant benefits to prosthetic dentistry. Various Intra-Oral Scanners (IOS) have been developed for mapping surface features of the mouth, largely using surface reflectance, including patterned light. The acquired surface contours of dental features enable computer-aided design and fabrication using CAD/CAM (Computer-Aided Design/Computer-Aided Manufacturing) tools. Some types of IOS have also been introduced to aid detection of caries, providing two-dimensional (2-D) surface information using fluorescence, near-IR, or transillumination imaging. Although there appears to be significant potential for improving dental procedures, however, with conventional 2-D surface scanning approaches there can be only limited information obtained about the dental object. As a result, IOS devices on the market remain somewhat limited in utility for dental clinics.

In more recent developments, a number of IOS devices have been disclosed for providing some level of depth-resolved imaging, using various types of signals that can penetrate to some depth below the surface in order to characterize the relative condition of teeth and soft tissue beneath the surface. These depth-resolved IOS systems employ non-ionizing energy, using, for example coherent light, ultrasound, photo-acoustics, or other energy types that can be generated and directed throughout the mouth without concern for radiation exposure to the patient or to the attending staff. These scanning devices' penetrative and non-ionizing attributes are highly desirable, but thus far have not been incorporated into dental workflow in a seamless fashion, which can allow them to significantly advance dental clinic productivity and patient care.

In conventional practice, a routine dental check-up is highly visual and tactile and requires high levels of training and expertise; however, these routine exams are limited primarily to surface conditions, lacking the capability to examine a lesion that lies beneath the surface. Thus, the diagnostic and screening efficacy of current practice can be somewhat disappointing.

Non-ionizing depth-resolved imaging technologies that offer some penetrative capabilities can help to assist the practitioner to locate and define intraoral lesions, supplementing the conventional visual procedure and surface imaging with at least some amount of sub-surface information, without the risks associated with radiographic imaging.

Although IOS employing depth-resolved imaging technologies such as Optical Coherence Tomography (OCT) and others appear to offer considerable potential for improving productivity and advancing patient care, there are still some barriers to overcome. Among problems that hamper broad acceptance and adaptation of this and other depth-resolved imaging technologies are practical considerations that include how the depth-resolved data can be easily acquired, how pathology detection can be rapidly performed using the acquired data, and how the diagnostic results can be accurately interpreted and appropriately rendered for the dental practitioner, within the narrow time window that is allowable for patient examination during routine hygiene or prior to treatment.

The intraoral environment presents considerable challenge for depth-resolved IOS scanning. Factors that complicate scanning include moisture and presence of liquid, obstructions from surrounding features in the narrow space, and difficulty in visualizing the actual angle and position.

It can be appreciated that strict spatial constraints of the intraoral cavity, patient comfort for breathing and swallowing, and the required patient positioning as seated for examination, necessarily limit the size and form factor of the scanning apparatus. Unlike depth-resolved images for the eyes or for other external surfaces of the anatomy, IOS scanners require manual placement and operation so that, among other difficulties, the same point on a tooth surface typically receives the penetrating scan signal repeated times and at different angles as the scan proceeds. There are few, if any, apparatus and methods available to support aiming and alignment in the constrained space; thus, the practitioner must pay careful attention to feedback information from the generated image rendering in order to determine whether or not there is sufficient scan content obtained for a particular region of interest.

Volume imaging for intraoral subjects adds even further complications. Classical 3-D reconstruction solutions have been developed for acquiring and processing projection data using equipment setup that demands strict geometric metrics, such as with the subject patient in a fixed position. The intraoral scan environment and normal patient jaw motion precludes such dependence on such strict geometry; coordinates for each scan must be computed relative to other detected features within the mouth. Thus, conventional 3-D acquisition and reconstruction methods do not apply or are, at best, very difficult to adapt to the intraoral scan environment.

Unlike surface imaging content, the acquired depth resolved data itself, with readings obtained at many hundreds of discrete points along the surface in OCT scanning, for example, does not provide a “video stream”. The raw OCT signal itself shows the interference characteristic between the outgoing signal and the return signal of reflected and scattered light and cannot be directly interpreted by the scanning operator or hygienist. Instead, each of the acquired depth-resolved signals must first be processed in order to extract the embodied information that is stored as high-frequency signal content in each reading; after this, the processed results can then be assembled using tomography techniques, then related to the surface contour in order to provide useful display for allowing assessment of the patient. Due, in part, to delayed processing times and to difficulties in presenting the obtained data in a meaningful manner, it is difficult to have the examination results readily available during the scan itself.

As a further complication, when tomography processing is completed on the depth-resolved data, there can be more information in the depth-resolved image content than can be effectively rendered at one time or in any one type of rendering. Further interpretation and analysis of depth-resolved data are often needed in order to obtain useful information related to both surface and near-surface conditions of the intraoral tissue. Without processing and supplemental guidance to support the image acquisition, the practitioner who performs or observes the scan may be unaware of much of the information that can be available from the depth-resolved imaging content.

By way of example, one dental condition that can be difficult to identify in OCT tomographic data relates to sub-surface cracks in the patient's tooth. This type of condition can be detected in OCT image signal content, but only a highly trained observer can detect this condition after careful manipulation of the rendered OCT content itself. Without assistance from efficient image processing and innovative analysis software, useful diagnostic information included in the processed results may not be readily accessible to the viewer until some time after the patient exam.

Thus, at least because of differences relating to how depth-related image content is acquired and synthesized, and difficulties in interpreting what the image content actually means, it can be appreciated that conventional IOS apparatus and methods, largely developed for surface image data only, are not adequate for supporting and capitalizing on the capabilities of intraoral tomographic scanning and that innovative tools and techniques would allow IOS devices that use OCT and other depth-resolved imaging techniques to be more useful to the practitioner for identifying a problem and providing a higher level of care to patients. Moreover, the capability to provide up-to-date diagnostic results and assessment along with 3-D surface contour during the IOS scan can help to significantly improve clinical workflow and productivity.

### SUMMARY OF THE INVENTION

An object of the present disclosure is to advance the art of intraoral scanning. An embodiment of the present disclosure particularly addresses the need for improved methods for acquiring, processing, and presenting surface and depth-resolved image content during the intraoral scan.

Another object of the present disclosure is to address, in whole or in part, at least the foregoing and other deficiencies in the related art. It is a related object of this application to provide, in whole or in part, at least the advantages described herein.

These objects are given only by way of illustrative example, and such objects may be exemplary of one or more embodiments of the application. Other desirable objectives and advantages inherently achieved by the disclosed methods may occur or become apparent to those skilled in the art. The invention is defined by the appended claims.

According to one aspect of the disclosure, there is provided a method for scanning an intraoral feature of a patient, the method repeating a sequence of:
(a) energizing a hand-held scanner to generate a penetrating scan signal for characterizing a surface contour and condition of at least a portion of underlying tissue of the intraoral feature;
(b) acquiring, at the hand-held scanner, returned energy from scans of the penetrating scan signal obtained at a plurality of adjacent locations along the intraoral feature surface;
(c) processing the returned signal energy from each of the plurality of adjacent scanned locations to obtain tomographic image content and to reconstruct, from the obtained tomographic image content, a 3-D model of the intraoral feature; and
(d) further processing the obtained tomographic image content to identify one or more normal or abnormal conditions for intraoral tissue at each of the plurality of adjacent scanned locations, and simultaneously during the scanning repeating a sequence of:
   (i) displaying the reconstructed 3-D model and updating the reconstructed 3-D model from the obtained tomographic image content as the hand-held scanner is moved along the surface of the intraoral feature; and
   (ii) reporting the one or more normal or abnormal conditions identified at scanned locations of the intraoral feature.

### BRIEF DESCRIPTION OF DRAWINGS

The foregoing and other objects, features, and advantages of the invention will be apparent from the following more particular description of the embodiments of the invention, as illustrated in the accompanying drawings. The elements of the drawings are not necessarily to scale relative to each other.

FIG. 1 is a schematic diagram that shows components of an intraoral scanning apparatus for image acquisition and display according to an embodiment of the present disclosure.

FIG. 2 is a logic flow diagram that shows a continuous looping sequence for obtaining, processing, and displaying depth-resolved scanned data and reporting diagnostic results during and following an intraoral scan.

FIG. 3 is a schematic diagram that shows the components of one type of OCT apparatus using a Mach-Zehnder interferometer (MZI) system.

FIG. 4A is a schematic diagram showing information acquired during scanning, using the OCT system.

FIG. 4B is a schematic diagram that shows how 3-D volume information is generated using the A-, B-, and C-scan data.

FIG. 5 is a flow diagram that shows a sequence for intraoral scanning using the OCT intraoral scanning apparatus of FIG. 1.

FIG. 6 is a schematic diagram that shows a sequence used for detecting a surface using OCT scan data.

FIG. 7 is a schematic diagram showing the role played by the Trained Neural Network (TNN) in detecting an object of interest.

FIG. 8A is a logic flow diagram that shows the first iterative procedure, an initial data handling step for crack detection training of the neural network.

FIG. 8B is a logic flow diagram that shows the use of test performance metrics and test data in building the model, labeled as a model generation step.

FIG. 9A is a graph that plots precision/recall results for a typical training set of images for OCT crack detection.

FIG. 9B shows computation of precision and recall quotients and IoU computation.

FIG. 10 shows an exemplary text listing that can display to report detected conditions.

FIG. 11 shows an exemplary cross-sectional image showing tooth conditions.

FIG. 12 shows an exemplary volume image with text annotation.

FIG. 13 shows an exemplary dental chart showing detected tooth conditions as reported by the system of the present disclosure.

FIG. 14 shows an alternate display with side-by-side image content.

FIG. 15 is a perspective view showing a 3-D volume model that can be displayed as a multilayered structure.

FIG. 16 shows the use of a user interface control for viewing the reconstructed 3-D volume model at various tissue depths.

### DETAILED DESCRIPTION OF THE INVENTION

The following is a detailed description of exemplary embodiments, reference being made to the drawings in which the same reference numerals identify the same elements of structure in each of the several figures.

Where they are used in the context of the present disclosure, the terms “first”, “second”, and so on, do not necessarily denote any ordinal, sequential, or priority relation, but are simply used to more clearly distinguish one step, element, or set of elements from another, unless specified otherwise.

In the context of the present disclosure, the term “scattered light” is used generally to include light that is reflected and backscattered from an object.

In the context of the present disclosure, the term “energizable” describes a component or device that is enabled to perform a function upon receiving power and, optionally, upon also receiving an enabling signal.

The term “actuable” has its conventional meaning, relating to a device or component that is capable of effecting an action in response to a stimulus, such as in response to an electrical signal, for example.

In the context of the present disclosure, the term “coupled” is intended to indicate a mechanical association, connection, relation, or linking, between two or more components, such that the disposition of one component affects the spatial disposition of a component to which it is coupled. For mechanical coupling, two components need not be in direct contact, but can be linked through one or more intermediary components.

The general term “scanner” relates to an optical system that is energizable to project a scanned light beam of light, such as broadband near-IR (BNIR) light that is directed to the tooth surface through a sample arm and acquired, as reflected and scattered light returned in the sample arm, for measuring interference with light from a reference arm used in OCT imaging of a surface. The term “scanner” can also refer to a scanning optical element, such as an actuable MEMS (micro-electromechanical systems) scanner, mirror, or mirror array, for example. The term “raster scanner” relates to the combination of hardware components that sequentially scan light toward uniformly spaced locations along a sample, as described in more detail subsequently.

The term “in signal communication” as used in the application means that two or more devices and/or components are capable of communicating with each other in at least one direction via signals that travel over some type of signal path. Signal communication can be wireless. The term “event” is used herein to indicate an action that causes a signal to be generated, such as an operator press of a scanner button, for example.

The term “highlighting” for a displayed feature has its conventional meaning as is understood to those skilled in the information and image display arts. In general, highlighting uses some form of localized display enhancement to attract the visual attention of the viewer. Highlighting a portion of an image, such as an individual organ, bone, or structure, or a path from one chamber to the next, for example, can be achieved in any of a number of ways, including, but not limited to, annotating, displaying a nearby or overlaying symbol, outlining or tracing, display in a different color or at a markedly different intensity or gray scale value than other image or information content, blinking or animation of a portion of a display, or display at higher sharpness or contrast.

The term “set”, as used herein, refers to a non-empty set, as the concept of a collection of elements or members of a set is widely understood in elementary mathematics. The term “subset”, unless otherwise explicitly stated, is used herein to refer to a non-empty proper subset, that is, to a subset of the larger set, having one or more members. For a set Q, a subset may comprise the complete set Q. A “proper subset” of set Q, however, is strictly contained in set Q and excludes at least one member of set Q.

The term “subject” refers to the tooth or other portion of a patient that is being imaged and, in optical terms, can be considered equivalent to the “object” of the corresponding imaging system. The term “lesion” has its conventional meaning, indicating a structural or composition change in an anatomical feature resulting from disease, infection, or injury.

Spatially relative terms, such as “beneath”, “below”, “lower”, “above”, “upper”, and the like, may be used herein for ease of description to describe one element or feature's relationship to another element(s) or feature(s) as described and illustrated in the figures. For a patient lying in a dental chair, relative geometry can be applied. Thus, it should be understood that terms that are spatially relative to the scanner and its emitted signals are intended to encompass different orientations of the scanner in use or operation in addition to the orientation depicted in the figures. For scanning within the mouth, for example, scanner orientation must track the local geometry of the intraoral features with relative position considered. Thus, for example, for upper teeth, lesions or objects that can be described herein as lying “beneath” the scanned surface may actually be positioned at a level to, or at a higher absolute spatial elevation in relation to, the scanned surface.

The term “tomography” as used herein relates to reconstruction of internal structure of a 3-dimensional (3-D) subject as a “volume”, according to information obtained from a penetrating energy. The information that is available for reconstruction can vary with the depth achievable from the penetrating energy. In conventional x-ray tomography, for example, the relatively high penetrating energy can enable tomography to generate a full volume image of a tooth, bone structure, or other dental feature.

For hand-held intraoral scanners as described herein, however, the penetrating energy for imaging is limited to non-ionizing energy, available from coherent light, ultrasound, photoacoustics, or other lower energy sources scanned into the subject. Thus, a tomographic volume can be generated, but the available image content that can be represented may not extend more than a few mm into the scanned subject. For intraoral features and conditions, however, depth-resolved information to 2-3 mm can be sufficient for generating a tomographic volume with features that can be observed and interpreted for diagnostic purposes. A slice of the tomographic volume displayed in cross section at any suitable angle can be similarly helpful for making diagnosis.

Apparatus and methods of the present disclosure can provide an IOS solution that employs a penetrative imaging technology, coupling 3-D surface scanning and detection with diagnostic screening in an integrated clinical workflow. This approach addresses the deficiencies of current commercial IOS devices and enables routine dental examination to proceed more efficiently and effectively, during the process of obtaining an intraoral scan, and using non-ionizing energy levels.

In the context of the present disclosure, the term “tissue” is used as in a biological sense, to connote anatomical structures formed naturally within the body from cellular aggregates, such as tooth, gums, skin, bone, and other such features. Tissue can be a complex biological structure; healthy tooth tissue itself, for example, comprises four major tissue types: enamel, cementum, dentin, and pulp. Embodiments of the present disclosure can help to characterize the condition of tissue along and below the surface of teeth, gums, and intraoral features in general. The condition of scanned tissue, on the surface and underlying the surface to some limited depth, can be characterized according to image content, for example, as sound or unsound, healthy, or diseased, normal, or abnormal, etc.

As one implementation of the Applicant's solution, Optical Coherence Tomography (OCT) is employed. OCT is a non-invasive, depth-resolved imaging technique that uses interferometric principles to obtain high resolution, tomographic images that characterize the depth structure of a sample. Particularly suitable for in vivo imaging of human tissue, OCT has shown its usefulness in a range of biomedical research and medical imaging applications, such as in ophthalmology, dermatology, oncology, and other fields, as well as in ear-nose-throat (ENT) and dental imaging. The description that follows describes an embodiment in terms of an exemplary IOS system that uses OCT technology.

OCT has been aptly described as a type of “optical ultrasound”, imaging reflected and scattered energy from within living tissue to obtain cross-sectional and volume data. In an OCT imaging system, light from a wide-bandwidth source, such as a super luminescent diode (SLD) or other low-coherence light source, is directed along two different optical paths: a reference arm or path of known optical path length and a sample arm or path that illuminates the tissue or other subject under study. Reflected and/or back-scattered light from the reference and sample arms can then be recombined in the OCT apparatus and the resulting interference effects can then be used to determine characteristics of the surface and near-surface underlying structure of the sample. Interference data can be acquired by rapidly scanning the illumination across the sample. At each of several thousand points along the sample surface, the OCT apparatus obtains an interference profile which can be used to reconstruct an A-scan, with an axial depth into the material that is largely a factor of light source and detection characteristics. For most tissue imaging applications, OCT can use broadband illumination sources and can provide image content at depths of up to a few millimeters (mm).

By comparison to the conventional 2-D environment of the x-ray image, with separate, well-defined stationary source and detector positions, surface and depth-resolved imaging data is acquired from the intraoral scanner of the embodiments described herein, as the scanner is manually moved along the mouth, translating from one point position to the next. The intraoral tomographic scan acquires depth data from the patient's mouth using a combination of reflected and backscattered light energy from each of potentially thousands of discrete locations along the intraoral surface. The energy levels used for dental OCT, for example, can obtain measurements to a nominal depth near about 2-3 mm in tooth material. Generating the contour of the tooth surface and making diagnostic sense of the scan information from the obtained tomographic data not only requires considerable processing and analysis of the returned light from each discrete location, but also entails the processing-intensive task of combining and organizing the resulting array of data so that it can be correlated with the actual intraoral surface contour of the patient's teeth, and can be displayed to the practitioner and accurately interpreted.

### Depth-Resolved Scanning

OCT, ultrasound, and opto-acoustic imaging technologies can each provide depth-resolved imaging from a suitably configured intraoral scanner. Each of these depth-resolved imaging modes direct a series of depth-penetrating output imaging signals into the scanned surface at a location, using generated signal energy that is capable of penetration beneath the surface of tissue to provide depth-resolved scanned data. Scanners operating in such depth-resolved imaging modes can obtain, in the same scan, both surface imaging data for surface contour reconstruction as well as subsurface information related to features that lie beneath the surface of the imaged tissue, accurate to some depth, depending in part on the limitations of the signal energy that is used and on characteristics of the scanned tissue itself. In addition to depth-related information, these depth-resolved scanning apparatus and approaches can also provide useful data on feature density, distribution, and dimension.

Embodiments of the present disclosure can use depth-resolved imaging to address the need for enhanced characterization of teeth and related tissue and other intraoral features during the intraoral scan. Using the added information obtained from depth-resolved scanned data, the Applicant's method adds dimension and further utility to the dental practitioner, supplementing the standard tooth-by-tooth assessment that is conventionally obtained by visual examination with display content that both characterizes the surface contour and highlights underlying features previously obscured or hidden below the surface and often inaccessible without the added cost, procedural complexity, and risks of radiographic imaging. Importantly, using apparatus and methods described herein, the capture and processing of diagnostic data does not require additional steps for the practitioner, hygienist, or other staff who perform the intraoral scanning workflow, because diagnostic functions can be performed within the time span of intraoral scanning itself. This capability for rapid assessment during the scan significantly increases the productivity of the dental practitioner and potentially leads to more accurate identification of conditions needing attention.

The description that follows focuses on OCT data acquisition for enhanced intraoral scanning and characterization of intraoral conditions during standard dental visits to a hygienist or other dental staff. It should be noted, however, that similar processing described herein can be used for reconstructing surface content with alternative depth-resolved scanning types that are capable of capturing data for generating tomographic volumes, such as when using ultrasound and photoacoustic imaging for example.

Advantageously, the depth-resolved scanned data acquired using the scanner according to the present disclosure contains both the information for reconstructing surface contour and the information on underlying tissue that is useful in assisting diagnosis of an intraoral region. The two types of information—surface and depth-resolved—are not acquired in separate operations, but are derived from the same scanned data at each location of the dental object. By simultaneously obtaining both surface and sub-surface image content contained within the same intraoral scan data, methods of the present disclosure address problems of spatial and temporal synchronization that had previously limited the utility, accuracy, and practicality of earlier depth-resolved and surface contour scanning solutions.

Embodiments of the present disclosure can utilize results acquired from an intraoral scanning apparatus that performs depth-resolved imaging such as optical coherence tomography (OCT) in order to alert the dental team to various conditions on and near the tooth surface. Because the scanner system is configured to provide detailed information on sub-surface conditions during the scanning session, apparatus and methods of the present disclosure can help to accelerate the response of the practitioner to identified conditions, such as fine cracks in and below the enamel surface for example, at an early stage. By utilizing the capabilities of depth-penetrating scanning for surface reconstruction and diagnosis, embodiments of the present disclosure can advance the art of dental care, with benefits to both the patient and the dental practice. Subsequent description gives more detailed information on such a scanner system according to an embodiment of the present disclosure.

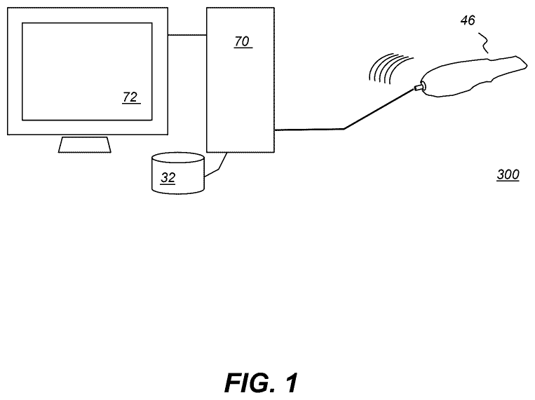

FIG. 1 is a schematic diagram that shows components of an intraoral scanning apparatus 300 for depth-resolved data acquisition that provides surface contour content and diagnostic information display, according to an embodiment of the present disclosure. An intraoral scanner (IOS) probe 46, can be energized to acquire depth-resolved scanned data, such as OCT data from reflected and back-scattered light. Probe 46 is in signal communication with support and signal processing circuitry for image processing and rendering logic in processor 70, which can be any suitable apparatus that provides programmable control processing logic, such as a computer. Signal communication with probe 46 and between components of apparatus 300 can be wired or wireless, or may use some combination of wired and wireless transfer. Processor 70 can be in signal communication with a memory or with other data storage 32, online remotely or on-site, that provides longer term data retention and archival. Display 72, also in signal communication with processor 70, can be used to render displayed image content and for command selection and entry.

Data obtained from probe 46 can include image data obtained using a reflectance light technique, as well as depth-resolved data, such as OCT data content for the same intraoral feature. Processor 70 logic can then process the different types of scanning results to detect conditions and render the needed imaging content on display 72.

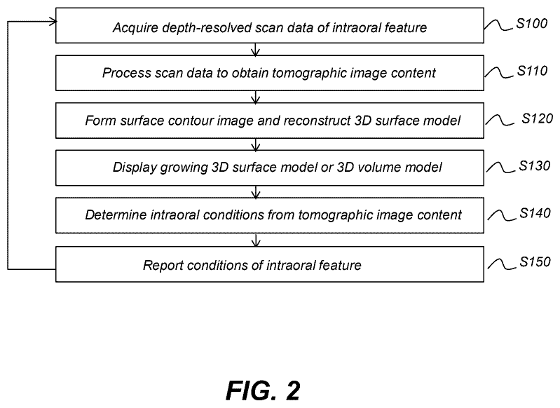

It would be useful to provide the depth-resolved image content and diagnostic results during the scanning session itself, preferably in “real-time”, that is, with results for a scanned portion of the mouth and diagnostic findings displayed as the scan is in process, with little or no perceptible delay to the scanning operator. To address this need, the logic flow diagram of FIG. 2 shows an overall, continuous looping sequence for obtaining, processing, and displaying depth-resolved scanned data and reporting diagnostic results during and following an intraoral scan, according to an embodiment of the present disclosure. An acquisition step S100 directs the penetrating scan signal at an intraoral object over a plurality of scan locations and obtains the returned signal energy through the same hand-held IOS device. A processing step S110 can then generate tomographic image content from each scan, using well-known methods for processing the depth-resolved scan data, as is described subsequently in more detail for OCT.

Continuing with the FIG. 2 sequence, a reconstruction step S120 can then extract surface information of the scanned object from the tomographic image content to form a surface contour image and to reconstruct, by an image stitching process that incrementally adds content from ongoing scan results, a dynamically growing 3-D surface model of the intraoral object. Stitching techniques for rendering surface contour and features using adjacent scans are familiar to those skilled in the tomographic and surface imaging art.

Following tomographic image content generation, 3-D volume reconstruction that includes underlying tissue can also be computed as part of, or following, step S120 in the processing sequence. Volume reconstruction, described and shown in more detail subsequently, can be used to form a 3-D volume model of the depth-resolved tissue, showing the outer surface layer of the teeth with underlying layers of tissue extending to a few mm beneath the surface, as constrained by the penetration distance of the OCT signal.

In a display step S130, the growing 3-D surface model or 3-D volume model is displayed and changed dynamically with updated data while the scan continues over subsequent portion of the mouth. A detection step S140 can use trained machine-learned logic to characterize one or more lesions, abnormal conditions, or other features of interest in the obtained tomographic volume while scanning continues. A reporting step S150 can then output detection results to a display for viewing by the operator or in some other form. The reporting output can be a text listing or can be a set of markers indicating conditions that have been detected, displayed on one or more portions of the tomographic image content. The output information can also be sent to data storage. Preferably, the sequence of steps S100 through S150 executes and repeats as scanner probe 46 is moved within the mouth, translated along intraoral surfaces in operation.

It should be noted that a number of the basic steps shown in FIG. 2 can be expanded significantly to include additional operations, depending on the system configuration. For example, acquisition step S100 can also include acquiring reflectance image content, such as color imaging or other 2-D image content that can be used for generating preview images or color texture for a point cloud, mesh, or other surface representation of the intraoral feature. Processing step S110 would then include executing the steps needed to align and map the generated surface contour, obtained from the tomographic image content, with the reflectance image content. Display step S130 can be expanded to include providing sections of the reflectance image and various views of the tomographic image content, including cross-section and volume image, along with the 3-D surface or 3-D volume model during scanning.

Notably, according to an embodiment of the present disclosure, both tomographic and surface contour image content can be obtained from the same depth-resolved scan signal, as described in more detail subsequently.

### Oct Scanning, Data Processing, and Image Display

As noted previously, OCT is one salient type of depth-resolved image acquisition and serves as an example method for describing an implementation of the intraoral scanner in an embodiment of the present disclosure.

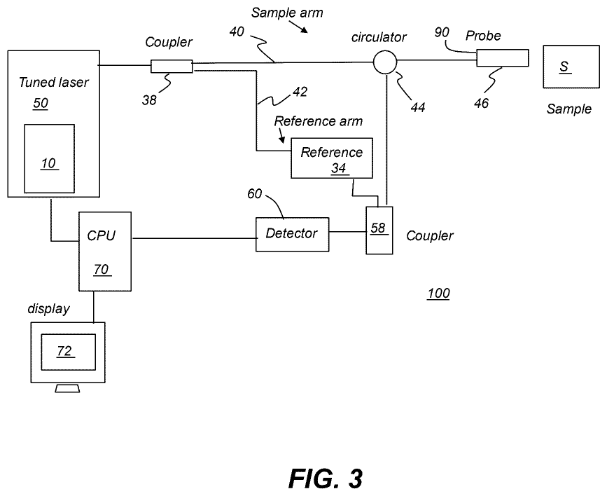

By way of example, the simplified schematic diagram of FIG. 3 shows the components of the penetrative intraoral scanner consisting of one exemplary type of OCT apparatus, here, a swept-source OCT (SS-OCT) apparatus 100 using a Mach-Zehnder interferometer (MZI) system. This type of OCT apparatus uses a wavelength-tunable light source provided by a wavelength filter 10 that is part of a tuned laser source 50, which can be a laser, super-luminescent light-emitting diode (LED), super-continuum light source, or other type of wide-bandwidth light source. For intraoral OCT, for example, laser source 50 can be tunable over a range of frequencies (expressed in terms of wave-numbers k) corresponding to wavelengths between about 400 and 1600 nm at a high sweep rate. According to an embodiment of the present disclosure, scanning over a tunable range of about 20 to 120 nm bandwidth centered about 1300 nm and with a frequency sweep rate from about 20 kHz to 2 GHz can be used for intraoral OCT. Other types of OCT apparatus can be used, including, but not limited to, time-domain OCT (TD-OCT) or spectral domain OCT (SD-OCT).

In the FIG. 3 device, the variable tuned laser 50 output goes through a coupler 38 and to a sample arm 40 and a reference arm 42. The sample arm 40 signal goes through a circulator 44 and is directed for scanning of a sample S by a scanning mirror 90 inside a handpiece or probe 46. The sampled signal is directed back through circulator 44 and to a detector 60 through a coupler 58. The reference arm 42 signal is directed by a reference 34, which can be a mirror or a light guide, through coupler 58 to detector 60. The detector 60 may use a pair of balanced photodetectors configured to cancel common-mode noise.

Control logic processor (control processing unit CPU) 70 is in signal communication with tuned laser 50 and its programmable filter 10 and with detector 60. Processor 70 can control the scanning function of probe 46 and store any needed calibration data for obtaining a linear response to scan signals. Processor 70 obtains and processes the output from detector 60. CPU 70 is also in signal communication with a display 72 for command entry and OCT results display.

It should be noted that the swept-source architecture of FIG. 3 is one example configuration only; there are a number of ways in which the interferometer components could be arranged for providing swept-source OCT scanning.

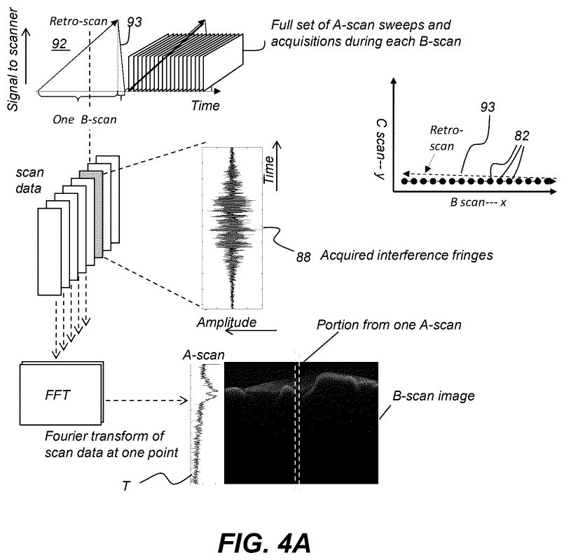

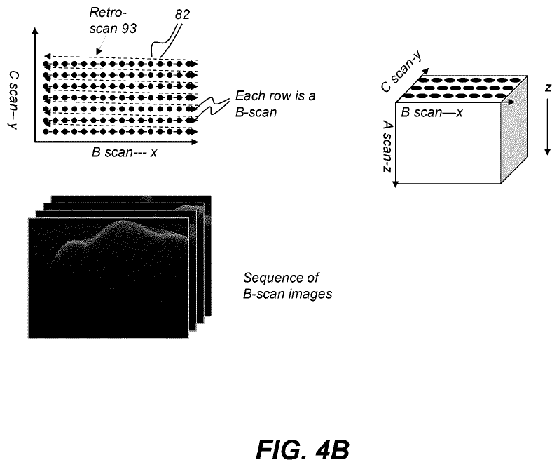

By way of further background, FIGS. 4A and 4B give an overview of the OCT scanning pattern as executed by probe 46. At each point in the scanning sequence, the OCT device obtains an A-scan from the acquired scan data. A linear succession of A-scans can then be aligned to form a B-scan, aligned to the x-axis direction as shown in FIG. 4A. Successive B-scan rows, side-by-side, then form a C-scan, one type of tomographic volume that provides the depth-resolved OCT image content for an intraoral sample S.

FIG. 4A schematically shows the information acquired during OCT scanning. The scan signal for obtaining each B-scan image has two linear sections in the example shown, with a scan portion 92, during which the scanning mirror is driven to direct the sampling beam from a beginning to an ending position, and a retro-scan 93, during which the scanning mirror is restored to its beginning position. An interference signal 88, shown with DC signal content removed, is acquired over the time interval for each point 82, wherein the signal is a function of the time interval required for the sweep, with the signal that is acquired indicative of the spectral interference fringes generated by combining the light from reference and feedback sample arms of the interferometer (FIG. 3). The Fourier transform FFT generates a transform T, which provides an A-scan corresponding to each point 82. One transform signal corresponding to an A-scan is shown by way of example in FIG. 4A.

From the above description, it can be appreciated that a significant amount of data is acquired over a single B-scan sequence. In order to process this data efficiently, a Fast-Fourier Transform (FFT) is used, transforming the time-based signal data to corresponding frequency-based data from which image content can more readily be generated.

In Fourier domain OCT, the A scan corresponds to the Fourier transform of one line of interference spectrum acquisition which generates a line of depth (z-axis) resolved OCT signal. The B scan data generates a 2-D OCT image along the corresponding scanned line. The B-scan image content represents a slice or cross-section of the 3-D tomographic volume obtained in OCT scanning.

Raster scanning is used to obtain multiple B-scan data by incrementing the raster scanner 90 acquisition in the C-scan (y-axis) direction. This is represented schematically in FIG. 4B, which shows how 3-D volume information (C-scan image content) is generated in stages using A-scans obtained from raster scanning in x and y directions.

It should be noted that the C-scan is a spatial arrangement of image signal content according to scan location. The C-scan data itself can thus be considered as an array of transform T signals (that is, A-scans) arranged along an x-y plane, as shown in FIG. 4B. Further processing can then be used to render the acquired C-scan data more suitable for viewing and interpretation, such as using rasterization or ray casting. With additional processing, the transform T signals generated from multiple C-scans can then be used to reconstruct a 3-D volume image of the scanned region. Tissue features within the 3-D volume image can be viewed from different perspectives, depicted in cross-section, and otherwise presented and manipulated on the display screen as a type of 3-D object for closer examination.

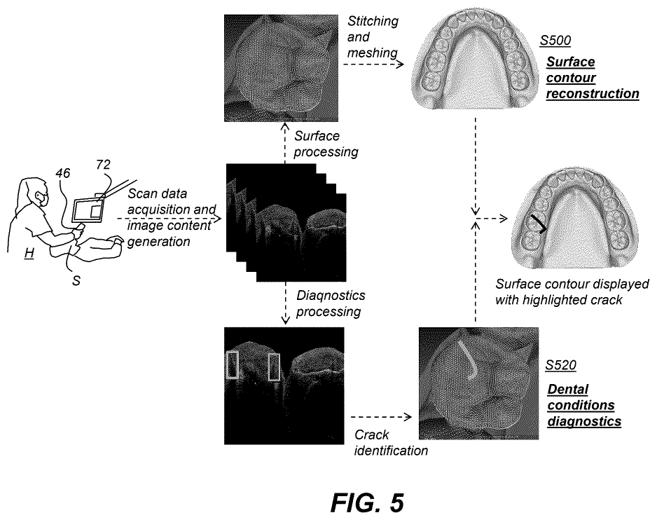

FIG. 5 is a diagram that shows a sequence for scanning an intraoral sample S using OCT intraoral scanning apparatus 300 of FIG. 1. As the scanner is translated along the intraoral sample, depth-resolved scan data is acquired from every scanned location of the sample and is processed to generate tomographic or volume image content of the sample. Two processing operations can be performed on the acquired tomographic image content as the scanning proceeds: a surface contour reconstruction step S500 and a dental conditions diagnostics step S520. A hygienist H, or practitioner or other dental staff member, can scan the patient using probe 46 while viewing results of the two operations of steps S500 and S520 on display 72. The growing reconstructed surface contour model and/or other image content, including 2-D color images, cross-sectional image, and OCT volume images from the current scanner position at sample S, can display, continually refreshed as the scanner is conveyed or translated from one position to the next within the mouth. Surface and near-surface diagnostics, provided using machine learning as described in more detail subsequently, are performed and reported as the scanning continues. In a preferred embodiment of the present disclosure, the diagnostic results can be marked on the displaying image content as highlight regions of particular concern that lie near the current scanner probe 46 position. The highlighting can appear on the 3-D surface model and/or other image content during or after scanning.

Because tomographic image content and dental diagnostics are processed from the same depth-resolved data, the diagnostics results can be readily mapped to the surface image content at each scanned point. Using this mapping allows the scanner system logic to highlight areas of diagnostic interest directly on the displayed image content as the scanning is executed. In the example of FIG. 5, crack detection is performed and a sub-surface crack location can be identified from the OCT data and mapped to its corresponding location on the surface contour reconstruction. The resulting displayed 3-D surface contour image shows surface and sub-surface condition of the tooth or other feature, as the scan proceeds.

### Extracting the Surface From Oct Data

As the patient's mouth is scanned, the OCT scanned data are obtained from light energy reflected and scattered by surfaces of teeth, gums, and other structures that lie beneath the surface, as deep as about 2-3 mm from the intraoral surface. As described previously, the OCT scanned data is processed to take the form of A-scan profiles. Numerous A-scans are obtained, extending into the sample from hundreds of surface points. B- and C-scans that incorporate the A-scan data provide sufficient information that can be extracted to form a point cloud or corresponding mesh that shows the contour of the surfaces of scanned sample S.

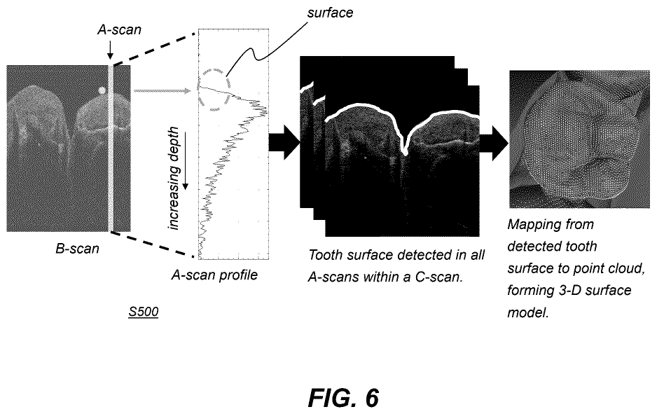

The schematic diagram of FIG. 6 shows steps in surface reconstruction step S500 (FIG. 5) for identifying a surface coordinate from OCT scanned data in order to form an accurate 3-D surface model of the patient's dentition. As shown in FIG. 6, the A-scan profile indicates a transition at some depth in the OCT scan signal. This transition corresponds to an interface between air and enamel and can be detected using methods such as gradient-based detection, for example. An exemplary detection metric would relate to the maximum positive gradient along the A-scan, as shown. The transition shown provides the surface location on the A-scan, which, through application of the known geometric calibration data of the OCT intraoral scanning apparatus, can be converted to a spatial coordinate for the surface point. The set of detected surface coordinate points along all A-scans that are included within a C-scan can then be mapped to form a spatially accurate 3-D point cloud.

Individual point clouds that employ these surface coordinate points to represent adjacent surface contour can be combined for 3-D surface model reconstruction by using a stitching process that involves properly aligning and joining together surface contour image content extracted from adjacent views that provide a data subset of the full set of acquired image content and wherein members of this data subset share adjacent features. Stitching and meshing algorithms for 3-D surface model reconstruction, well-known to those skilled in the imaging arts, enable the processor to reconstruct a continuous surface from multiple scans. The reconstructed 3-D surface model of the scanned sample can be displayed in realistic color by texture mapping, using 2-D color images captured for each view during scanning. Texture mapping techniques are well-known to those skilled in the imaging arts.

The 3-D surface model reconstructed from combined surface coordinates can be used for display as a point cloud or mesh, as well as to design prosthetic devices. According to an embodiment of the present disclosure, point cloud coordinate data can be organized as a type of Computer-Aided Design/Manufacturing (CAD/CAM) design file for forming prosthetic or other dental devices. The file can be an STL (STereo Lithography) file, commonly used in additive manufacture or 3-D printing and other CAM machining applications, for example.

### Detection Using Machine Learning

Identification of particular intraoral conditions from visual or tactile examinations, particularly for sub-surface conditions such as sub-surface cracks or caries for example, can be difficult, even to the trained eye of a dental specialist. An intraoral scan using the apparatus and method of the present disclosure during routine dental check-ups can help to improve preliminary assessment of tooth conditions by employing a trained neural network to assess the tomographic image content generated from the acquired depth-resolved scan data.

Typically, machine learning techniques employ multi-layer neural network architectures and can employ associated techniques that detect higher-level patterns from a volume of low-level data such as “deep learning”, for example. In the context of the present disclosure, terms such as “machine learning”, “artificial intelligence”, “deep learning”, and “neural networks” can be used equivalently to describe trained logic from various related aspects.

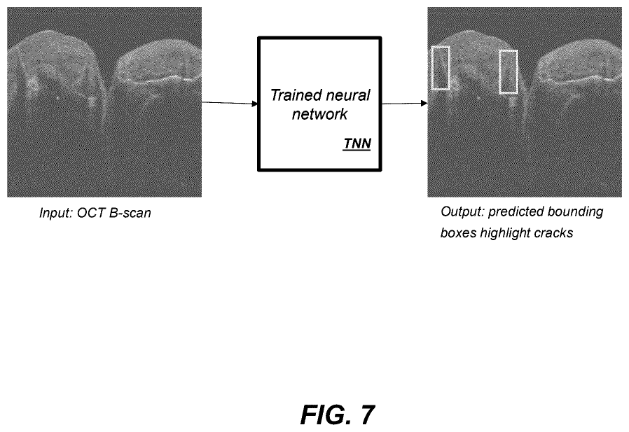

The simplified schematic of FIG. 7 shows the role played by the Trained Neural Network (TNN) in detecting an object of interest according to embodiments of the present disclosure, shown for the example of sub-surface cracks in the tooth enamel. The TNN applies a mathematical (statistical) model, generated from a training sequence, to the processing problem. Input to the processing is the OCT A-scan, B-scan, or C-scan volume, assembled from a sequence of point scans or A-scans, as described previously. The TNN is trained to predict and localize predefined lesion or other objects, such as cracks, or other classes from the OCT A-scan, B-scan, or C-scan image.

The identified lesion from TNN logic can then be highlighted on the display in an appropriate manner, such as by outlining as shown in FIG. 7, or using color, animation, or other image enhancement. The resulting output rendered to display 72 (FIG. 1) can denote each detected crack, fissure, or other feature, such as employing the bounding boxes shown.

### Generating the Mathematical Model—Training Sequence for the Neural Network

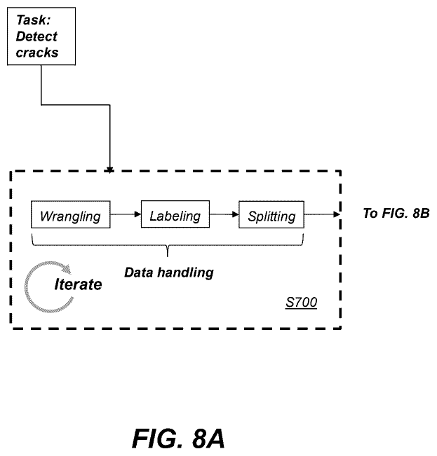

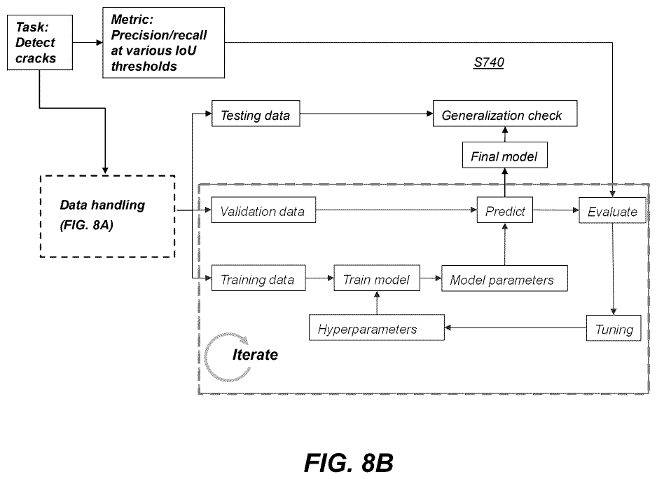

In order to provide the trained logic, a training sequence is executed, in which test images that have been identified as having a particular condition are analyzed by the software according to a model having multiple characteristics. The logic flow diagram given in FIGS. 8A and 8B shows an exemplary training pipeline that can be used for detecting conditions such as sub-surface cracks in the example used herein. A similar sequence can be applied for identifying other conditions of interest in surface and sub-surface intraoral scanning.

Training for this function can be a supervised process and involves the repetitive sequence of submitting A-scan, B-scan, or C-scan image renderings to the neural network, wherein some portion of the submitted renderings (considered positive) are identified as positive and indicate various lesions and features of interest for system identification; other submitted images (considered negative) show only normal structure and features. As the network training proceeds, correct identification improves and false positive/false negative conclusions become increasingly less likely, until satisfactory results are achieved.

The training process can be considered to involve two iterative procedures that cooperate to build the model that drives neural network logic. FIG. 8A shows the first iterative procedure, an initial data handling step S700 for crack detection. In data handling, in-vivo OCT scan data is collected using the intraoral scanning probe 46 of the present disclosure and processed to provide the data as image content. The wrangling process is a data organization step that involves selection of the feature set that is best suited to the assigned task. Given this feature set, appropriate utilities for standardized processing can be determined. In performing labeling, the processed data is partitioned into images that have cracks and images that do not have cracks. The splitting step is composed of splitting the data into three sets: training dataset, validation dataset, and testing dataset.

To label the features of interest for neural network detection, bounding boxes can be applied as labels. These labels can identify and localize the cracks within the image and identify (label) this feature, as shown in FIG. 7. This dataset of images and bounding box labels can then be used to train the TNN model. In general, the more labeled and diverse the data for training, the better the model performance.

FIG. 8B shows the use of test performance metrics and test data in building the model, labeled as a model generation step S740. Metrics are applied in order to test model predictions to determine the relative performance between different models and to give a sense of how the models will perform in practice. The test dataset is carefully selected and should come from a separate collection of data that is not related to the training dataset, in order to reduce testing bias.

As shown in FIG. 8B, the training data set is directed along an iterative path that generates a trained model, providing model parameters, and generating a prediction for each training image. Validation data provides ground truth information that enables evaluation of the prediction and generation of tuning adjustment and hyperparameters, which are attributes of the model or even the training process that can be adjusted to improve performance, such as the number of layers within the model, or loss function, or even data augmentation. The main purpose for the validation dataset is to ensure that the model is not overfitting to the training dataset. The model is configured to work efficiently in a real-world environment and with real-time performance.

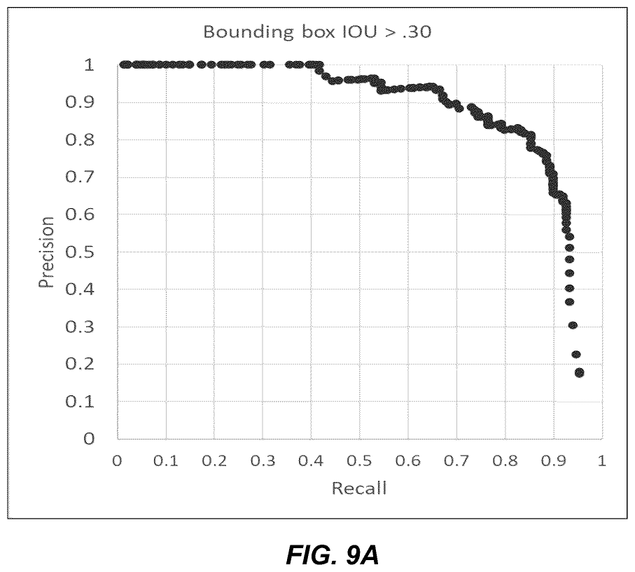

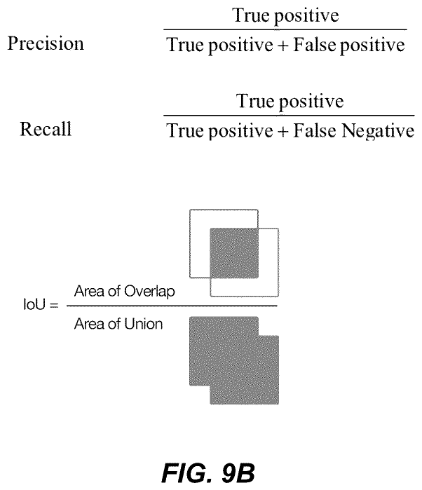

A primary performance metric that is widely used is the intersection over union (IoU) based precision recall curves, as shown in the example of FIG. 9A. An Intersection over Union (IoU) threshold helps to evaluate the accuracy of a generated model by indicating the overlap between a ground truth region and a prediction region. The IoU threshold value is a number between 0 and 1, quantifying a ratio between areas of overlap and areas of union for model and ground truth, as illustrated conceptually in FIG. 9B. In object detection, IoU quantifies how closely the prediction of a model overlaps ground truth for a particular object in an image, mediated by the relative amount of error in the prediction. An IoU value of 1 indicates perfect overlap between the model and ground truth; the 0 value indicates no measurable overlap.

When the computed IoU is above a predefined threshold, the predicted box is considered a positive identification; below the threshold indicates a negative identification. From these results, precision and recall scores can be constructed, whether a true positive, false positive, or false negative. FIG. 9A shows an example precision/recall curve that plots results for a typical training set of images for tooth crack detection using the intraoral scanning apparatus of the present disclosure, using an exemplary threshold value of 0.30. Computation of precision and recall quotients is shown, for example, in FIG. 9B.

### Reporting Detected Conditions

Intraoral scanning apparatus 300 of FIG. 1 is capable of assisting identification of intraoral features, lesions, and abnormal dental conditions of interest to the dental practitioner, including, but not limited to:
(i) surface and sub-surface cracks in enamel;
(ii) composite filling defects;
(iii) enamel caries;
(iv) secondary caries;
(v) caries beneath sealant;
(vi) enamel demineralization;
(vii) plaque or calculus;
(viii) erosion or abrasion; and

### (ix) Gum Inflammation.

After performing dental diagnostic assessment based on the acquired tomographic image content, intraoral scanning apparatus 300 can report all conditions which it has identified, determined according to system logic. Reporting of detected features as well as normal or abnormal conditions can be performed in a number of ways, including but not limited to the reporting methods shown in subsequent examples.

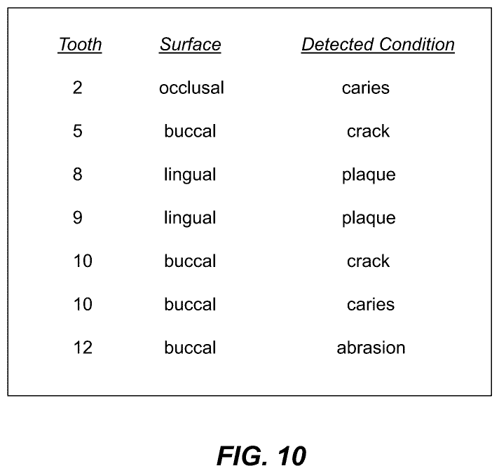

As scanning proceeds, any identified pathological or other abnormal conditions can be displayed in simple text format, listing relevant information such as a corresponding tooth number and a tooth surface, as shown in FIG. 10. Entries in the list can be added as the scanner is conveyed over different intraoral regions and finds new conditions of interest. A text file can readily be displayed, stored, or provided in printed form.

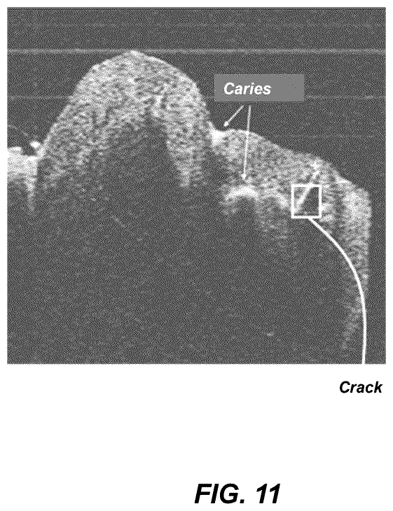

Alternately, detected conditions can be displayed, with annotation and highlighting, including various types of symbols, arrows, bounding boxes, heat maps, or other markers, which may display in different colors on a point cloud or cross-sectional rendering as shown in the example of FIG. 11. The cross-sectional image and pathology labeling can appear momentarily when pathological conditions are initially identified or can be continuously updated as the scanner is conveyed along the teeth and new conditions are sensed.

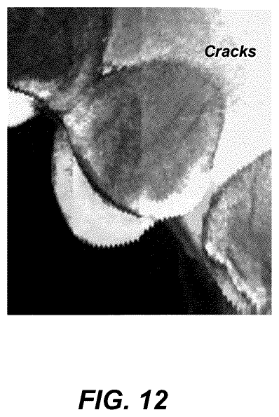

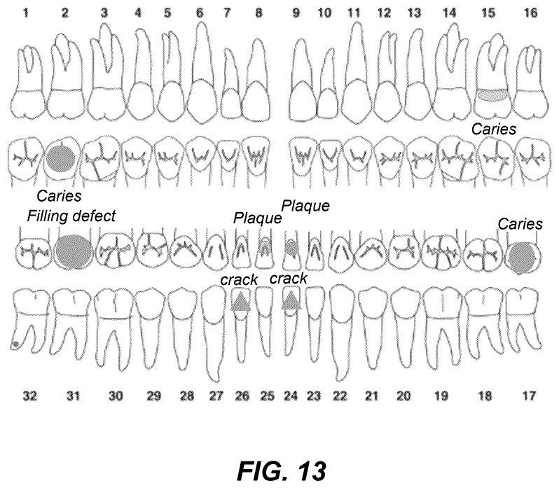

Alternately, the detected conditions can be highlighted, such as using arrows, bounding boxes, heat maps, or other markers and with different colors, on a volume image or C-scan, as shown in FIG. 12, with text annotation. The volume image with highlighting and labeling can appear when pathological conditions are found, and can be continuously updated as the scanner scans the teeth and identifies new conditions. Similarly, the detected conditions can be highlighted on a teeth map or dental chart, such as that shown in FIG. 13, with supplemental text annotations. Highlighting and labels can be added to the teeth map or chart as various conditions are detected, with the annotation continuously updated as additional scan data is acquired.

Detected conditions can be highlighted, such as using arrows, bounding boxes, heat maps, or other markers and with different colors, on a growing 3-D surface contour model, such as that described with reference to FIG. 5, with text annotations. Highlighting and labeling can continuously update as the scanner scans the teeth and identifies new features or tissue conditions. The processor can transmit at least a portion of the reconstructed 3-D surface contour image to prosthetic design software

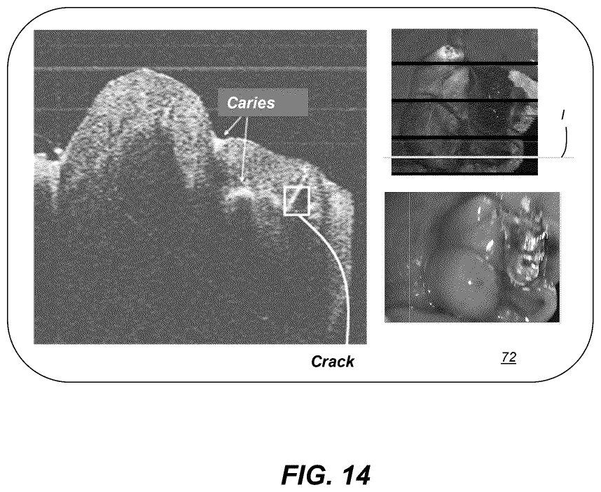

According to an alternate embodiment of the present disclosure, one or more reporting methods can be employed, displaying results side-by-side. FIG. 14 shows a set of images that can be generated according to an embodiment of the present disclosure, and that can be included simultaneously on the same display 72 screen, for example: a tomographic slice with caries identified, a top view showing a reconstructed surface, and a top view of a reflectance image for a tooth. A sub-surface crack can also be highlighted in this image set. A slice index I can be manipulated by the operator in order to change the cross-sectional slice of the tomographic volume that displays. Index I can be extended in orthogonal directions, such as spanning or at right angles to the B-scan rows. Alternately, index I can be used to show a cross-section through the C-scan volume at angles oblique to the B-scan rows of data. In addition to being displayed in real-time during scanning, any of the above described reports can be saved to data storage in electronic form or printed as a hard copy report.

### 3-D Model and Multilayer Visualization

Embodiments of the present disclosure can generate two related types of 3-D models from the OCT scan data. A 3-D surface model, as the term implies, provides a 3-D representation of the surface contour for a scanned intraoral feature. Once it is generated using C-scan data, by stitching together coordinates from adjacent point clouds employing surface points extracted from individual A-scans, the 3-D surface model is a single-layer representation of the surface of the intraoral feature, without any image content for tissue that lies beneath the surface.

The second type of 3-D model is a 3-D volume model. Unlike the 3-D surface model that is obtained from points of the C-scan that correspond only to the surface layer of an intraoral feature, the 3-D volume model incorporates the complete C-scan content, including all of the depth-resolved image content of the intraoral feature.

One approach for reconstructing a 3-D volume model from OCT scan data uses direct stitching of adjacent C-scans; relative to the sequence shown in FIG. 2, this stitching process can be executed after tomographic image content is generated in Step S110. Although this method is feasible, it can require extensive processing since, prior to stitching, individual OCT light paths in the C-scan must first be corrected to compensate for optical path changes due to changes in the refractive index of the scanned tissue. But complete correction of optical paths within the tissue is not possible because the refractive index is neither constant nor completely known. Hence, spatial accuracy for subsurface points within each corrected C-scan can be difficult to achieve. Further, a stitching process that simply combines adjacent C-scans together for volume reconstruction compounds spatial errors and can cause inaccuracy in generating the 3-D volume model. The generated 3-D volume model can be further voxelized for 3-D visualization and rendering using methods well known in the 3-D image processing arts.

Thus, in order to reconstruct a 3-D volume model of the scanned intraoral feature with improved spatial accuracy, an embodiment of the present disclosure uses an alternate approach. The Applicant's strategy can be carried out following reconstruction of the 3-D surface model in Step S120 of the FIG. 2 sequence. The 3-D surface model generated in step S120 is a highly accurate representation of the surface of the scanned intraoral feature because it is the result of stitching together only the extracted surface points of neighboring or adjacent C-scans. This is then used as a basis structure for spatial positioning of the A-scan associated with each point of the 3-D surface model to form a 3-D volume model. Subsequently, the position of points along the A-scan, at varying depths beneath the surface, can then be corrected by accounting for optical path changes due to the refractive index of the subsurface tissue. Importantly, in this alternative approach, subsurface points of each A-scan are not involved in the stitching process; thus, any residual spatial error from incomplete optical path correction of these subsurface points is not compounded in the generation of the 3-D volume model. The generated 3-D volume model, formed by associating A-scan data with the 3-D surface model, can be further voxelized using methods well known in 3-D image processing art for 3-D visualization and rendering.

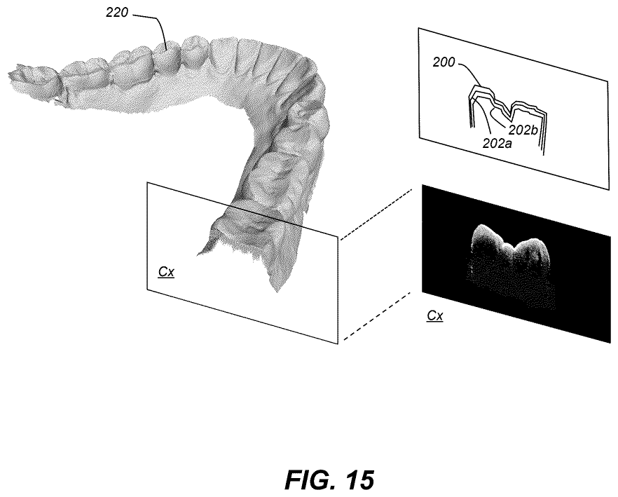

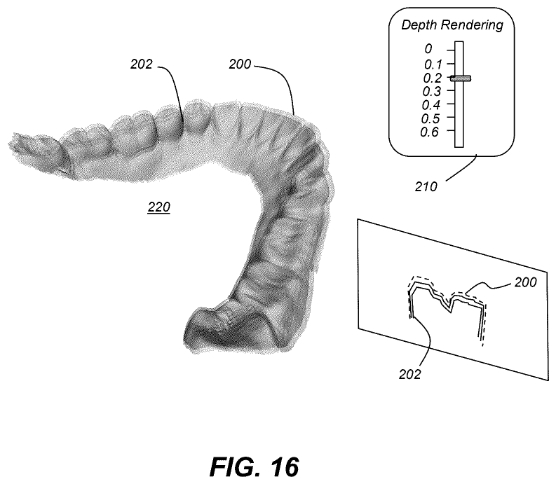

By way of example, FIGS. 15 and 16 show an exemplary processed 3-D volume model generated from the scan signals.

As shown in FIG. 15, a reconstructed 3-D volume model 220 of a scanned dental arch can be rendered for display as a multilayered structure that not only shows a surface layer 200, but also includes voxels that can be rendered for selective display of underlying tissue, stacked or layered beneath the surface along the track of each A-scan. In this way, voxels corresponding to underlying tissue can be considered as lying in one or more underlying layers 202, with layer assignment depending only on relative voxel distance to surface layer 200; this distance corresponds to depth from the surface. Thus, for example, with a surface layer of voxels considered to be surface layer 200, the voxels that are immediately adjacent to the surface layer 200 voxels can be considered to constitute a first underlying layer 202a. Following this pattern, a second underlying layer 202b can be rendered using those voxels immediately adjacent to the first underlying layer 202a, and so on for successively increasing depths. Each successive layer can be rendered as points with equal depth distance from the actual surface of the intraoral feature.

Using the methods just described for generating the 3-D volume model allows the system to provide a multi-layer rendering of the reconstructed depth-resolved volume image. Surface layer 200 would have the thickness of a single voxel; successive underlying layers 202 could have a user-designated thickness and depth, such as a given distance dimension from surface layer 200.

In FIG. 15, a cross-section view Cx shows a basic arrangement of depth-resolved 3-D volume model 220 with surface layer 200 corresponding to the 3-D surface model and one or more underlying layers 202a, 202b, etc. The total number and depth of hidden, underlying layers 202 is effectively constrained by the depth-resolution that is available to the penetrating signal used by the scanner. For intraoral OCT scanning, for example, the resolvable depth is generally no more than about 2-4 mm. Each underlying layer 202 can simply be the set of those voxels that represent tissue that lies within a certain depth distance or range of depth distances from the surface 200.

The surface 200 of 3-D volume model 220 gives the same representation of the scanned arch as the 3-D surface model. 3-D volume model 220 can also provide visibility of the underlying layers 202. To allow visibility to inner, underlying tissue layers, FIG. 16 shows the use of a user interface control 210, such as a manual or on-screen slidebar, for viewing selective portions or layers of volume model 220 at different layer depths. In the exemplary image of 3-D volume model 220 that is shown, surface layer 200 (e.g. Depth Rendering slidebar value “0”) is “stripped away” and is either not visible or is largely transparent, such as shown in dim outline or with reduced opacity. Successive underlying layers 202, each containing voxels within a given range of depths determined by an entered default or preset value or using user interface control 210, can thus be viewed, such as by an operation that strips or peels away the outer surface 200 and zero or more underlying layers 202 of the scanned dentition or a region of interest within the scanned dentition. Multi-layer visualization can be provided for the whole or for a part of the 3-D volume model.

According to an embodiment of the present disclosure, the resolution for layers can be configured or adjusted to selectively display a desired depth of the volume reconstruction. This allows, for example, determining the depth of a tooth crack or other feature or abnormal condition of intraoral tissue, so that the extent of the intraoral feature can be accurately ascertained during or following the scan.

During the OCT scan, the display can indicate an area where a particular condition is probable, based on results from the neural network. This indication can enable or encourage the operator to return to the problem area and perform additional scanning or examination in the highlighted region, including peeling back successive layers, as shown in the example of FIG. 16. Annotation and labeling of a lesion or other abnormal condition can also be helpful, using results from TNN evaluation. The scanning system can also prompt the hygienist or other operator to revisit or concentrate on a particular tooth or area, such as by providing a message or an indication on-screen, for example. Because the OCT scan has a 2-3 mm depth, obtaining sub-surface scan content with the scanner deployed at a number of different angles can be beneficial.

The machine learning logic itself can execute on any computer processor that is capable of the needed data access, computational, and storage resources required for the application, including “cloud” or graphical processing unit or GPU processing, for example. In addition to display at the operator console, an embodiment of the present disclosure also makes it possible to generate a printed report on at least one surface or sub-surface lesion identified in the tomographic volume.

The invention has been described in detail, and may have been described with particular reference to a suitable or presently preferred embodiment, but it will be understood that variations and modifications can be effected within the spirit and scope of the invention. The presently disclosed embodiments are therefore considered in all respects to be illustrative and not restrictive. The scope of invention is indicated by the appended claims, and all changes that come within the meaning and range of equivalents thereof are intended to be embraced therein.

1. A method for scanning an intraoral feature of a patient, the method repeating a sequence of:
(a) energizing a hand-held scanner to generate a penetrating scan signal for characterizing a surface contour and condition of at least a portion of underlying tissue of the intraoral feature;
(b) acquiring, at the hand-held scanner, returned energy from scans of the penetrating scan signal obtained at a plurality of adjacent locations along the intraoral feature surface;
(c) processing the returned signal energy from each of the plurality of adjacent scanned locations to obtain tomographic image content and to reconstruct, from the obtained tomographic image content, a 3-D model of the intraoral feature; and
(d) further processing the obtained tomographic image content to identify one or more normal or abnormal conditions for intraoral tissue at each of the plurality of adjacent scanned locations, and simultaneously during the scanning repeating a sequence of:
(i) displaying the reconstructed 3-D model and updating the reconstructed 3-D model from the obtained tomographic image content as the hand-held scanner is moved along the surface of the intraoral feature; and
(ii) reporting the one or more normal or abnormal conditions identified at scanned locations of the intraoral feature.

2. The method of claim 1 wherein the 3-D model is either:
(i) a 3-D surface model generated as a surface layer of voxels having assigned coordinates; or
(ii) a 3-D volume model that contains depth-resolved image content of the intraoral feature, wherein the depth-resolved image content is formed by stitching together the tomographic image content obtained at adjacent scanned locations or by associating the obtained tomographic image content with corresponding points on the generated 3-D surface model.

3. The method of claim 1 wherein the penetrating signal is an ultrasound signal or a photoacoustic signal.

4. The method of claim 1 wherein the penetrating signal is an optical coherence tomography signal from a laser or from a light-emitting diode.

5. The method of claim 1 wherein the returned signal energy comprises back-scattered and reflected light.

6. The method of claim 1 wherein reconstructing the 3-D model comprises correcting one or more optical paths within the intraoral feature for signal content.

7. The method of claim 1 wherein processing the returned signal energy comprises using machine learning logic that is trained to identify at least one abnormal condition for tissue that is on or beneath the scanned surface.

8. The method of claim 1 further comprising labeling an identified condition in surface or underlying tissue on the display of the reconstructed 3-D model.

9. The method of claim 1 further comprising transferring at least a portion of the reconstructed 3-D model to dental design software.

10. The method of claim 1 further comprising providing a user interface utility that displays image content for tissue that lies beneath the surface of the 3-D model.

11. A method for characterizing the condition of an intraoral feature of a patient, the method repeating a sequence of:
(a) energizing a hand-held optical coherence tomography (OCT) scanner that directs a penetrating signal toward the intraoral feature of the patient at a plurality of scan locations and that acquires returned signal energy from each corresponding scan location;
(b) processing the returned signal energy from the OCT scanner to obtain tomographic image content from adjacent positions along the intraoral feature;
(c) reconstructing and rendering for display a three-dimensional (3-D) surface model from the obtained tomographic image content;
(d) repeatedly refreshing the 3-D surface model rendering according to subsequent scans of the feature surface;
(e) processing the obtained tomographic image content to characterize the intraoral feature condition; and
(f) reporting one or more characterized conditions of the intraoral feature as the scanner is conveyed along the intraoral feature.

12. The method of claim 11 further comprising reconstructing a 3-D volume model by correlating the reconstructed 3-D surface model with the obtained tomographic image content or stitching together the tomographic image content obtained at adjacent scanned locations.

13. The method of claim 11 wherein processing the obtained tomographic image content comprises using machine learning logic that is trained to identify at least one abnormal condition on or beneath the scanned feature surface.

14. The method of claim 11 wherein reporting consists of highlighting one or more of the identified abnormal conditions within the obtained tomographic image content or the rendered reconstructed 3-D surface model.

15. The method of claim 11 wherein reporting comprises storing the reporting results in electronic form.

16. The method of claim 12 further comprising providing an operator interface that accepts operator instructions for specifying the rendered portion of the reconstructed 3-D volume model according to depth from the surface.

17. The method of claim 11 further comprising generating a textual report on at least intraoral feature condition identified in the tomographic image content.

18. The method of claim 11 further comprising generating a dental chart or mapping that shows at least one surface or sub-surface condition identified in the tomographic volume.

19. The method of claim 11 further comprising providing diagnostic information related to the intraoral feature.

20. An apparatus for intraoral imaging comprising:
(a) a hand-held scanner that is energizable to direct a penetrating signal to an intraoral feature and to acquire returned signal energy;
(b) a processor configured to:
(i) energize the hand-held scanner to generate the penetrating signal;
(ii) acquire the returned signal energy from a plurality of adjacent scan locations;
(iii) process the returned signal energy from each of the plurality of adjacent scanned locations to obtain tomographic image content and to reconstruct, from the obtained tomographic image content, a 3-D model of the intraoral feature;
(iv) process the obtained tomographic image content to identify one or more normal or abnormal conditions for intraoral tissue at the corresponding scan location and, simultaneously:
display the reconstructed 3-D model that is continually updated from the obtained tomographic image content as the hand-held scanner is moved along the surface of the intraoral feature; and
report the one or more conditions identified in scan locations of the intraoral feature that is being scanned;
and
(c) a display in signal communication with the processor and configured to render images.
